(19) United States
(12) Patent Application Publication
(10) Pub. No.: US 2026/0103466 A1
(43) Pub. Date: Apr. 16, 2026

(54) PROCESS FOR THE MANUFACTURE OF SALTS AND CRYSTALLINE FORMS OF 1-(8-BROMOPYRIDO[2,3-E][1,2,4]TRIAZOLO[4,3-A]PYRAZIN-4-YL)-NMETHYLAZETIDIN-3-AMINE AND NOVEL CRYSTALLINE FORMS

(71) Applicant: JW PHARMACEUTICAL CORPORATION, Gyeonggi-do (KR)

(72) Inventors: Kim TROENSEGAARD NIELSEN, Ballerup (DK); Flemming HOFMANN LARSEN, Ballerup (DK); Anders KLARSKOV PETERSEN, Ballerup (DK)

(21) Appl. No.: 19/115,875

(22) PCT Filed: Sep. 28, 2023
(86) PCT No.: PCT/EP2023/076913

§ 371 Date: Mar. 27, 2025

(30) Foreign Application Priority Data
Sep. 29, 2022 (EP) 22198611.0

(51) Int. Cl. C07D 471/14

(52) U.S. Cl. CPC C07D 471/14

(57) ABSTRACT

A novel process for preparation of crystalline forms and novel crystalline forms of 1-(8-bromopyrido[2,3-e][1,2,4]triaz olo[4,3-a]pyrazin-4-yl)-N-methylazetidin-3-amine salts is provided along with pharmaceutical compositions comprising the same. Also disclosed is the use of the novel polymorph for the treatment of diseases, such as atopic dermatitis (AD), itch, pruritus and various forms of urticaria for example chronic idiopathic urticaria subtypes.

### SUMMARY OF THE INVENTION

The present invention relates to a novel process for the preparation of pharmaceutically acceptable salts of the compound of formula (I)

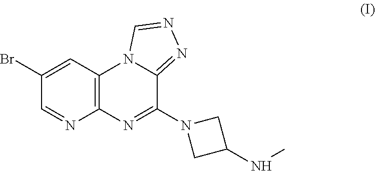 [SMILES: CNC1CN(c2nc3ncc(Br)cc3n3cnnc23)C1]

using specific salts or crystalline forms of 1-(8-bromopyrido[2,3-e][1,2,4]triazolo[4,3-a]pyrazin-4-yl)-N-methylazetidin-3-amine.

The present invention also relates to novel crystalline forms of 1-(8-bromopyrido[2,3-e][1,2,4]triazolo[4,3-a]pyrazin-4-yl)-N-methylazetidin-3-amine methyl sulfate and ethyl sulfate and pharmaceutical compositions comprising the novel crystalline forms and to the use of the novel crystalline forms for the treatment of diseases, such as atopic dermatitis (AD), itch, pruritus and various forms of urticaria for example chronic idiopathic urticaria subtypes, such as cholinergic urticaria. Also provided herein is a method of preparing the crystalline form of the invention.

### BACKGROUND OF THE INVENTION

U.S. Pat. No. 9,586,959 relates, among other compounds, to the compound 1-(8-bromopyrido[2,3-e][1,2,4]triazolo[4,3-a]pyrazin-4-yl)-N-methylazetidin-3-amine and pharmaceutically acceptable salts thereof, processes for preparing such compound as well as pharmaceutical compositions comprising the same. The patent discloses the preparation of a number of salts of the compound 1-(8-bromopyrido[2,3-e][1,2,4]triazolo[4,3-a]pyrazin-4-yl)-N-methylazetidin-3-amine.

1-(8-bromopyrido[2,3-e][1,2,4]triazolo[4,3-a]pyrazin-4-yl)-N-methylazetidin-3-amine exhibits a strong histamine 4 receptor inhibitory effect and shows suppressive effects against histamine induced infiltration of inflammatory cells such as mast cells and eosinophils. The compound thus has strong anti-inflammatory and anti-itching effects and may therefore be useful for treating a range of diseases such as those disclosed in U.S. Pat. No. 9,586,959, including AD.

Different crystalline solid forms of chemical compounds may have distinct physical properties such as e.g. chemical stability, physical stability, hygroscopicity, melting point, solubility, dissolution rate, morphology and bioavailability which make them more or less suitable as the selected active ingredient in a pharmaceutical product.

In addition, a chemical entity may exist in several different crystalline solid forms and these include different polymorphic forms that share the same sum formula (e.g. anhydrates) and different solvates (e.g. hemihydrate, monohydrate and dihydrates) of the same chemical entity which do not share the same sum formula. Such crystalline solid forms have distinct crystal structures and vary in physical properties as described above. The different crystalline solid forms can be distinguished from each other by e.g. melting point, XRPD pattern, spectral characteristics (e.g. FT-IR, Raman and SS-NMR), and other physical and chemical properties. Chemical entities can also exist in amorphous form.

The actual crystalline form selected therefore plays an important role in the development and manufacture of an active pharmaceutical ingredient. Should a single crystal form be required, it is important that the crystallization process be robust and reliably produce the desired crystalline form in polymorphically pure form and that the crystalline form does not change (e.g. interconvert to a different crystalline form) during the relevant manufacturing processes, and/or during storage.

A number of different salts of 1-(8-bromopyrido[2,3-e][1,2,4]triazolo[4,3-a]pyrazin-4-yl)-N-methylazetidine have been identified and disclosed in U.S. Pat. No. 9,586,959.

Some of the salts appear as anhydrate, others as monohydrates and dihydrates each in several polymorphic forms that interconvert upon drying or loses water at relatively low temperature and they are therefore not suitable for development as a pharmaceutical.

The invention also relates to novel crystal forms of 1-(8-bromopyrido[2,3-e][1,2,4]triazolo[4,3-a]pyrazin-4-yl)-N-methylazetidine salts having beneficial properties making the suitable as API in pharmaceutical products and in the manufacture if other suitable salts and crystal forms of 1-(8-bromopyrido[2,3-e][1,2,4]triazolo[4,3-a]pyrazin-4-yl)-N-methylazetidin-3-amine.

It has been found the 1-(8-bromopyrido[2,3-e][1,2,4]triazolo[4,3-a]pyrazin-4-yl)-N-methylazetidin-3-amine methyl sulfate salt including form A and B and the ethyl has a very high solubility in solvents that are suitable for pharmaceutical production which makes the salt particularly useful for purification by recrystallisation and for preparation of other crystalline salts of the compound of formula (I).

Other crystalline forms such as 1-(8-bromopyrido[2,3-e][1,2,4]triazolo[4,3-a]pyrazin-4-yl)-N-methylazetidin-3-amine ethyl sulfate and 1-(8-bromopyrido[2,3-e][1,2,4]triazolo[4,3-a]pyrazin-4-yl)-N-methylazetidin-3-amine methane sulfonate also have similarly useful properties in relation to purification by recrystallisation and for preparation of other crystalline salts of the compound of formula (I).

### SUMMARY OF THE INVENTION

Accordingly the present invention relates to process for the preparation of the compound of formula (I)

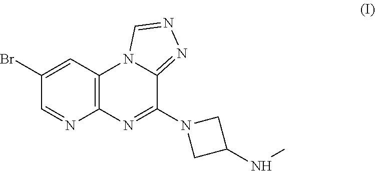 [SMILES: CNC1CN(c2nc3ncc(Br)cc3n3cnnc23)C1]

or a pharmaceutically acceptable acid addition salt thereof comprising
A. preparing crystalline form of methyl sulfate salt, the ethyl sulfate salt and/or the methane sulfonate salt of the compound of formula (I) by
   i. obtaining a crystalline form of the methyl hydrogen sulfate salt, the ethyl sulfate and/or the methane sulfonate of the compound of formula (I);
   ii. optionally followed recrystallization of the crystalline form of the methyl sulfate salt, the ethyl sulfate salt and/or the methane sulfonate salt of the compound of formula (I) in a suitable solvent or solvent mixture; and
B. dissolving the crystalline form of the methyl sulfate salt, the ethyl sulfate salt and/or the methane sulfonate salt of the compound of formula (I) in a suitable solvent or solvent mixture followed by addition of an acid capable forming a pharmaceutically acceptable acid addition salt of the compound of formula (I), or
dissolving the crystalline form of methyl sulfate salt, ethyl sulfate salt and/or the methane sulfonate salt of the compound of formula (I) in a suitable solvent or solvent mixture comprising an acid capable of forming a pharmaceutically acceptable acid addition salt of the compound of formula (I);
C. crystalizing said acid addition salt of the compound of formula (I) or the free base of the compound of formula (I) by cooling the reaction mixture or by adding an antisolvent;
D. optionally followed by recrystallization and isolation of the acid addition salt of the compound of formula (I) or the free base of formula (I) from a suitable solvent.

The present invention also relates to crystalline form A of 1-(8-bromopyrido[2,3-e][1,2,4]triazolo[4,3-a]pyrazin-4-yl)-N-methylazetidin-3-amine methyl sulfate monohydrate characterized by one or more XRPD reflections at approximately (° 2θ) 8.9, 12.3 and/or 15.7 (±0.2 degrees).

The present invention also relates to crystalline from B of 1-(8-bromopyrido[2,3-e][1,2,4]triazolo[4,3-a]pyrazin-4-yl)-N-methylazetidin-3-amine methyl sulfate anhydrate characterized by one or more XRPD reflections at approximately (° 2θ) 8.1, 14.9 and/or 26.5 (±0.2 degrees).

The present invention also relates to crystalline from a of 1-(8-bromopyrido[2,3-e][1,2,4]triazolo[4,3-a]pyrazin-4-yl)-N-methylazetidin-3-amine ethyl sulfate form A characterized by one or more XRPD reflections at approximately, characterized by one or more XRPD reflections at approximately 8.1, 14.6 and/or 23.4 (±0.2 degrees).

The invention also relates to a pharmaceutical composition comprising the novel crystalline forms mentioned above and a pharmaceutically acceptable carrier.

In one embodiment the invention relates to a novel crystalline form or pharmaceutical composition comprising a novel crystalline form for the treatment of disease selected from atopic dermatitis, itch, pruritus, and various forms of urticaria, including chronic idiopathic urticaria subtypes.

The invention also relates to a pharmaceutical composition comprising the crystalline forms mentioned above and a pharmaceutically acceptable carrier.

In one embodiment the invention relates to a compound or pharmaceutical composition as described above for the treatment of disease selected from atopic dermatitis, itch, pruritus, and various forms of urticaria, including chronic idiopathic urticaria subtypes.

### DETAILED DESCRIPTION OF THE INVENTION

The 1-(8-bromopyrido[2,3-e][1,2,4]triazolo[4,3-a]pyrazin-4-yl)-N-methylazetidin-3-amine methyl sulfate salt, 1-(8-bromopyrido[2,3-e][1,2,4]triazolo[4,3-a]pyrazin-4-yl)-N-methylazetidin-3-amine ethyl sulfate salt and 1-(8-bromopyrido[2,3-e][1,2,4]triazolo[4,3-a]pyrazin-4-yl)-N-methylazetidin-3-amine methane sulfonate salt has been found to be particularly useful for crystallization, recrystallization and for purification or for crystallization of other salt forms of the same compound due to their larger solubility in solvents useful for crystallization and recrystallization purposes in the manufacture of API for pharmaceutical use.

Compared to the 1-(8-bromopyrido[2,3-e][1,2,4]triazolo[4,3-a]pyrazin-4-yl)-N-methylazetidin-3-amine hydrogen sulfate salt disclosed in U.S. Pat. No. 9,586,959 the methyl sulfate salt, ethyl sulfate salt and the methane sulfonate salt of 1-(8-bromopyrido[2,3-e][1,2,4]triazolo[4,3-a]pyrazin-4-yl)-N-methylazetidin-3-amine have a more neutral pH in aqueous environment and are therefore much more stable towards hydrolysis of the 1-(8-bromopyrido[2,3-e][1,2,4]triazolo[4,3-a]pyrazin-4-yl)-N-methylazetidin-3-amine molecule during processing in aqueous even at elevated temperatures at least as high as 50° C. This means that loss of API during manufacture can be reduced by using the methyl sulfate salt, ethyl sulfate salt and/or the methane sulfonate salt.

The process of the invention may be used to prepare any pharmaceutically acceptable acid addition salt of the compound of formula (I). Suitable pharmaceutically acceptable acid addition salts formed by reaction with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic fumaric acid or any other suitable acid suitable in pharmaceutical products.

In some embodiments the acid addition salt of the compound of formula (I) is a “hemi salt”, such as for example the hemisuccinate. Hemi salt forms are typically prepared by crystallization from a suitable solvent or solvent mixture comprising around one-half equivalent acid per equivalent compound of formula (I), such that the ratio between free base and acid is significantly different that 1:1. In the case of a hemi form comprising a di-acid (such as succinic acid), the ratio between the free base and the acid is approximately 1:0.5.

According to one embodiment, the invention relates to a process as above, wherein the crystalline form of 1-(8-bromopyrido[2,3-e][1,2,4]triazolo[4,3-a]pyrazin-4-yl)-N-methylazetidin-3-amine methyl sulfate is obtained by a process comprising:
i. dissolving a compound of formula (I) or a salt thereof in a suitable solvent or solvent mixture comprising methanol followed by addition of sulfuric acid, or dissolving a compound of formula (I) or a salt thereof in a suitable solvent or solvent mixture comprising methanol and methyl hydrogen sulfate;
ii. crystallizing the methyl sulfate salt of the compound of formula (I) by cooling the reaction mixture and/or adding an antisolvent;
iii. optionally followed recrystallizing and isolation of a crystalline form of the methyl hydrogen sulfate salt of the compound of formula (I) in a suitable solvent or solvent mixture.

According to one embodiment, the invention relates to a process as above, wherein the crystalline form of 1-(8-bromopyrido[2,3-e][1,2,4]triazolo[4,3-a]pyrazin-4-yl)-N-methylazetidin-3-amine ethyl sulfate is obtained by a process comprising
i. dissolving a compound of formula (I) or a salt thereof in a suitable solvent or solvent mixture comprising ethanol followed by addition of sulfuric acid, or dissolving a compound of formula (I) or a salt thereof in a suitable solvent or solvent mixture comprising ethanol and ethyl hydrogen sulfate;
ii. crystallizing the ethyl sulfate salt of the compound of formula (I) by cooling the reaction mixture and/or adding an antisolvent;
iii. optionally followed recrystallizing and isolation of a crystalline form of the ethyl sulfate salt of the compound of formula (I) in a suitable solvent or solvent mixture.

According to one embodiment, the invention relates to a process as above, wherein the ccrystalline form of 1-(8-bromopyrido[2,3-e][1,2,4]triazolo[4,3-a]pyrazin-4-yl)-N-methylazetidin-3-amine methane sulfonate is obtained by a method comprising:
i. dissolving a compound of formula (I) or a salt thereof in a suitable solvent or solvent mixture followed by addition of methane sulfonic acid, or dissolving a compound of formula (I) or a salt thereof in a suitable solvent or solvent mixture and methane sulfonic acid;
ii. crystallizing the methane sulfonate of the compound of formula (I) by cooling the reaction mixture and/or adding an antisolvent;
iii. optionally followed recrystallizing and isolation of the crystalline form of methane sulphonate of the compound of formula (I) in a suitable solvent or solvent mixture.

According to one embodiment, the invention relates to a process as above, wherein the solvent used in step i) and iii) is selected from methanol or a mixture of methanol and water and wherein an optional antisolvent is isopropanol or acetone.

According to one embodiment, the invention relates to a process as above, wherein the acid used in step i) and iii) is selected from ethanol or a mixture of ethanol and water and wherein an optional antisolvent is isopropanol or acetone.

According to one embodiment, the invention relates to a process as above, wherein the solvent used in step i) and iii) is selected from methanol or a mixture of methanol and water and wherein an optional antisolvent is isopropanol or acetone.

According to one embodiment, the invention relates to a process as above, wherein the crystalline form of 1-(8-bromopyrido[2,3-e][1,2,4]triazolo[4,3-a]pyrazin-4-yl)-N-methylazetidin-3-amine methyl sulfate is obtained by a method comprising deprotecting a compound of formula (II) in a suitable solvent or mixture of solvents comprising methyl hydrogen sulfate:

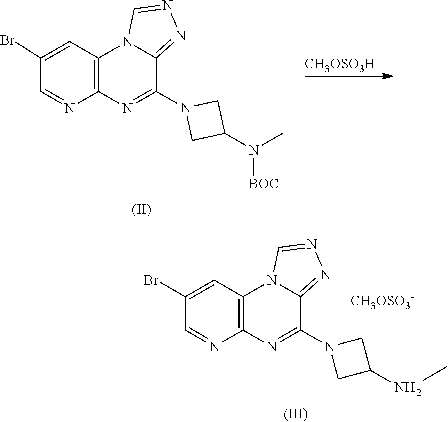

followed by crystallisation of 1-(8-bromopyrido[2,3-e][1,2,4]triazolo[4,3-a]pyrazin-4-yl)-N-methylazetidin-3-amine methyl sulfate by cooling the reaction mixture and/or by adding an antisolvent. Hydrates, of 1-(8-bromopyrido[2,3-e][1,2,4]triazolo[4,3-a]pyrazin-4-yl)-N-methylazetidin-3-amine methyl sulfate form A which is a monohydrate may be formed by adding water to the reaction mixture.

According to one embodiment, the invention relates to a process as above, wherein the crystalline form of 1-(8-bromopyrido[2,3-e][1,2,4]triazolo[4,3-a]pyrazin-4-yl)-N-methylazetidin-3-amine methane sulfonate is obtained by a method comprising deprotecting a compound of formula (II) in a suitable solvent or mixture of solvents comprising methane sulfonic acid:

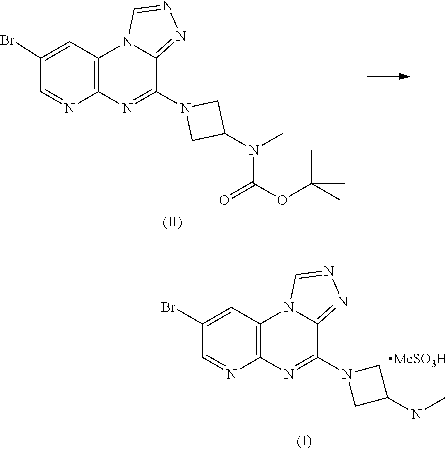 [SMILES: CN(C(=O)OC(C)(C)C)C1CN(c2nc3ncc(Br)cc3n3cnnc23)C1 ; "·MeSO3H" ; "(II)" ; CNC1CN(c2nc3ncc(Br)cc3n3cnnc23)C1 ; "(I)"]

followed by crystallisation of 1-(8-bromopyrido[2,3-e][1,2,4]triazolo[4,3-a]pyrazin-4-yl)-N-methylazetidin-3-amine methyl sulfate by cooling the reaction mixture and/or by adding an antisolvent.

According to one embodiment, the invention relates to a process as above, wherein the crystalline form of 1-(8-bromopyrido[2,3-e][1,2,4]triazolo[4,3-a]pyrazin-4-yl)-N-methylazetidin-3-amine methane sulfonate is obtained by a method comprising ring-closure of a compound of formula (III) to obtain a compound of formula (II)

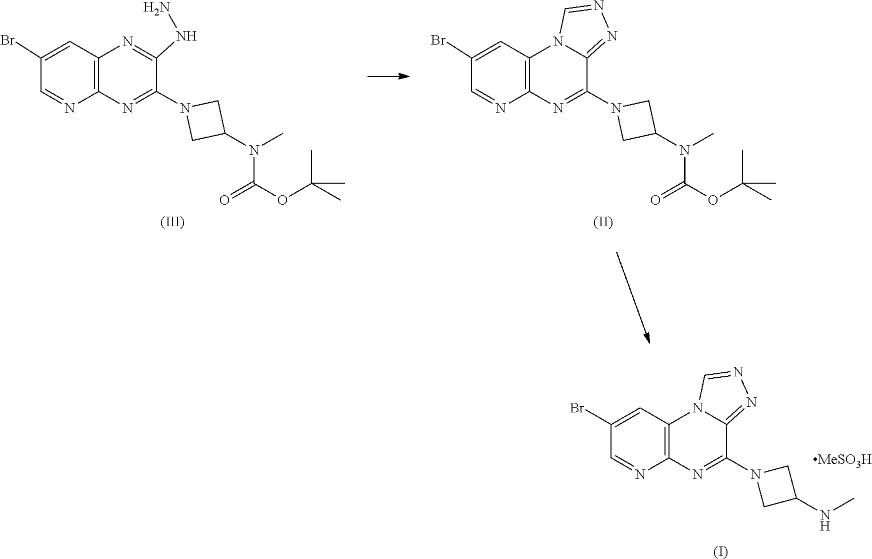

followed by crystallisation of 1-(8-bromopyrido[2,3-e][1,2,4]triazolo[4,3-a]pyrazin-4-yl)-N-methylazetidin-3-amine methyl sulfate by cooling the reaction mixture and/or by adding an antisolvent.

According to one embodiment, the invention relates to a process as above, wherein the pharmaceutically acceptable acid addition salt of the compound of formula (I) that is obtained in step D. is crystalline 1-(8-bromopyrido[2,3-e][1,2,4]triazolo[4,3-a]pyrazin-4-yl)-N-methylazetidin-3-amine hemisuccinate, suitably in the form of the monohydrate.

According to one embodiment, the invention relates to a process as above, wherein the solvent used for dissolving the methyl hydrogen sulfate salt, the methanesulfonate and/or the ethyl hydrogen sulfate salt of the compound of formula (I) in step B. is water or a solvent mixture containing water.

According to one embodiment, the invention relates to a process as above, wherein the antisolvent in step in step C. is acetone and/or isopropyl alcohol.

Two crystalline forms of 1-(8-bromopyrido[2,3-e][1,2,4]triazolo[4,3-a]pyrazin-4-yl)-N-methylazetidin-3-amine methyl sulfate have been identified. One form (form A) is a monohydrate and the other form (form B) is an anhydrate.

One crystalline form of 1-(8-bromopyrido[2,3-e][1,2,4]triazolo[4,3-a]pyrazin-4-yl)-N-methylazetidin-3-amine ethyl sulfate have been identified. Form A is an anhydrate.

The technical problem underlying this present invention is to circumvent the drawbacks of other crystalline and/or amorphous forms of 1-(8-bromopyrido[2,3-e][1,2,4]triazolo[4,3-a]pyrazin-4-yl)-N-methylazetidin-3-amine such as, solubility, ability to form crystals, filtration properties, solubility, thermodynamic properties, stability issues (e.g. due to water uptake), density, and transformation (e.g. interconversion to other polymorphic forms or hydrates/anhydrates) at varying degrees of humidity and during crystallization processes.

A total of 98 different crystallization experiments covering a broad range of organic solvents resulted in 8 different polymorphic forms (A-H) of the 1-(8-bromopyrido[2,3-e][1,2,4]triazolo[4,3-a]pyrazin-4-yl)-N-methylazetidin-3-amine methyl sulfate.

Among these pure forms of A, B, E, G, and H were obtained, but only form A and B were stable during gentle drying and attempts to prepare larger amounts of pure form E and G were not successful.

The novel crystalline forms according to the invention are methyl sulfate salts i.e. a salt form where there for each molecule of methyl sulfate is one molecule of 1-(8-bromopyrido[2,3-e][1,2,4]triazolo[4,3-a]pyrazin-4-yl)-N-methylazetidine in the crystal lattice.

Two crystalline forms of 1-(8-bromopyrido[2,3-e][1,2,4]triazolo[4,3-a]pyrazin-4-yl)-N-methylazetidin-3-amine methyl sulfate has been identified. One form (form A) is a monohydrate and the other form (form B) is an anhydrate.

Form A is a channel hydrate and generally channel hydrates/solvates are not very stable since the solvent molecules easily can move in and out of the channels and causes collapse of the crystal lattice.

By analysis of crystal form packing based on single crystal X-ray analysis it was observed that the water molecules in form A are located in channels. Such hydrates are known as non-stoichiometric hydrates and the location of water in channels can quite often cause stability issues. However, in form A the channels in which the water molecules are situated are small (3.2 Å on the shortest diameter and 5.4 Å on the longest diameter respectively) which makes it difficult to remove the water from the crystal lattice and thereby improved stability during processing.

In the case of form A the water molecules are therefore placed in narrow channels which makes it difficult for the water to escape which stabilizes the hydrate and makes form A particularly useful for use in a solid pharmaceutical product, such as in a tablet. Furthermore, form A can be dried under drying conditions such as freeze dryer or in a vacuum oven at 60° C. without collapsing the crystal lattice. Also form A has been found suitable for large scale drying.

By analysis of crystal form packing of form B based on single crystal X-ray analysis reveals that the packing of form B is a low symmetric triclinic packing stabilized by π stacking of the heteroaromatic moiety along all three axes (a, b and c) with the counterion situated between planes of the free base. Despite the smaller number of molecules in the unit cell and smaller volume of the unit cell of form B when compared to form A the density of both crystal forms is the same. Form B is stable in even in environments containing 60% water.

Moreover, the 1-(8-bromopyrido[2,3-e][1,2,4]triazolo[4,3-a]pyrazin-4-yl)-N-methylazetidin-3-amine methyl sulfate salt was found to have a relatively high solubility in a number of solvents, which makes the 1-(8-bromopyrido[2,3-e][1,2,4]triazolo[4,3-a]pyrazin-4-yl)-N-methylazetidin-3-amine methyl sulfate salt particularly useful for purification by recrystallisation and as a starting material for formation of any other salt of 1-(8-bromopyrido[2,3-e][1,2,4]triazolo[4,3-a]pyrazin-4-yl)-N-methylazetidin-3-amine, such as for example the of 1-(8-bromopyrido[2,3-e][1,2,4]triazolo[4,3-a]pyrazin-4-yl)-N-methylazetidin-3-amine hemisuccinate monohydrate. Other salts of 1-(8-bromopyrido[2,3-e][1,2,4]triazolo[4,3-a]pyrazin-4-yl)-N-methylazetidin-3-amine generally have relatively low solubility.

The solubility of 1-(8-bromopyrido[2,3-e][1,2,4]triazolo[4,3-a]pyrazin-4-yl)-N-methylazetidin-3-amine methyl sulfate salt is shown in table 1:

TABLE 1
Solubility of 1-(8-bromopyrido[2,3-e][1,2,4]triazolo[4,3-
a]pyrazin-4-yl)-N-methylazetidin-3-amine methyl sulfate
salt in various solvents

| Solvent | Solubility (μg/ml) | Solid form |
|---|---|---|
| Acetone | <3 | B |
| EtOAc | <3 | B |
| Methyl ethyl ketone | <3 | B |
| MTBE | <3 | A + B |
| THF | <3 | B |
| Toluene | <3 | A + B |
| Isoamyl alcohol | 6.3 | A + B |
| 2-propanol | 16 | A + B |
| 1-propanol | 50 | B |
| Acetonitrile | 55 | B |
| EtOH | 110 | B |
| MeOH | 1600 | B |
| DMF | 8200 | B |
| 1-propanol/H₂O | 44000 | A + B |
| DMSO | >/=69000 | A + C |

As may be seen in table 1 the solubility is very low in THF, EtOAC, Toluene, MTBE and methyl ethyl ketone, low in isoamyl alcohol and 2-propanol, intermediate in 1-propanol acetonitrile and ethanol, high in methanol and DMF and very high in 1-propanol:H₂O (1:5) and DMSO.

The solubility of 1-(8-bromopyrido[2,3-e][1,2,4]triazolo[4,3-a]pyrazin-4-yl)-N-methylazetidin-3-amine ethyl sulfate salt form A is shown in table 2:

TABLE 2
Solubility of 1-(8-bromopyrido[2,3-e][1,2,4]triazolo[4,3-a]pyrazin-
4-yl)-N-methylazetidin-3-amine ethyl sulfate salt in various solvents

| Solvent | Solubility μg/ml |
|---|---|
| EtOAc | <3.5 |
| Toluene | <3.5 |
| MTBE | <3.5 |
| Cyclohexane | <3.5 |
| DCM | <3.5 |
| 2-propanol | 4.7 |
| Acetone | 6.2 |
| Isoamyl alcohol | 23 |
| 1-propanol | 37 |
| Acetonitrile | 45 |
| EtOH | 120 |
| Methyl ethyl ketone | 200 |
| MeOH | 1600 |
| DMF | 5900 |
| 1,3-dimethyl-2-imidazolidinone | 17000 |
| DMSO | 76000 |

### Definitions

As used herein the term “rt” or “room temperature” indicates that the applied temperature is not critical and that no exact temperature value have to be kept. Usually, “rt” or “room temperature” is understood to mean temperatures of about 15° C. to about 25° C. [see e.g. EU Pharmacopoeia 7.5, 1.2 (2012)].

The term “solvate” as used herein describes a crystalline compound in which solvent molecules are incorporated into the crystal lattice of the compound in a stoichiometric or non-stoichiometric manner. If the solvent molecules are water the term “hydrate” is used herein.

The type of hydrate depends on the molar ratio of water molecules to 1-(8-bromopyrido[2,3-e][1,2,4]triazolo[4,3-a]pyrazin-4-yl)-N-methylazetidin-3-amine methyl sulfate molecules.

The term “monohydrate” implies 0.8 to 1.2 mol water per mol of 1-(8-bromopyrido[2,3-e][1,2,4]triazolo[4,3-a]pyrazin-4-yl)-N-methylazetidin-3-amine methyl sulfate.

An anhydrate is a crystal form which do not contain any water in the crystal lattice.

The term “non-hygroscopic” as used herein indicates that the increase in mass of a drug substance between about 0% to 80% relative humidity is less than 0.2% by weight.

In the context of the present invention, the term “XRPD reflection peak” denotes a particular 2θ position in an XRPD pattern, wherein the signal-to-noise ratio (calculated according to item 2.2.46 of the European Pharmacopoeia) is greater than 3/1. “Absence of a peak” is herein defined as a peak having an intensity of at most 1%, such as 0.5% or 0.2%, of the highest peak in an XRPD of a sample of the compound of the invention, i.e no detectable XRPD peak above background signals.

In an XRPD pattern, the main characteristics of diffraction line profiles are 2θ position, peak height, peak area and shape (characterized by, for example, peak width or asymmetry, analytical function, empirical representation). The 2θ position is the most important factor as for example the intensity will be affected by sample preparation, and the width of the peaks by particle size. In addition to the diffraction peaks, an X-ray diffraction experiment also generates a more-or-less uniform background in an XRPD pattern, upon which the peaks are superimposed. Besides specimen preparation, other factors contribute to the background, for instance the sample holder, diffuse scattering from air and equipment, other instrumental parameters such as detector noise, general radiation from the X-ray tube, etc. The peak-to-background ratio can be increased by minimizing background and/or by choosing prolonged exposure times.

### Abbreviations

DSC: Differential Scanning calorimetry
DVS: Dynamic Vapor Sorption
TGA: Thermogravimetric Analysis
XRPD: X-ray Powder Diffraction
¹³C CP/MAS NMR: ¹³C cross polarization magic angle spinning nuclear magnetic resonance
SXRD: Single crystal X-ray Diffraction

### BRIEF DESCRIPTION OF THE DRAWINGS

FIG. 1A: XRPD pattern for methyl sulfate form A (3-45° 2theta).

FIG. 1B: XRPD pattern for methyl sulfate form A (3-30° 2theta).

FIG. 2A: XRPD pattern for methyl sulfate form B (3-45° 2theta).

FIG. 2B: XRPD pattern for methyl sulfate form B (3-30° 2theta).

FIG. 3: DSC and TGA curve of methyl sulfate form A.

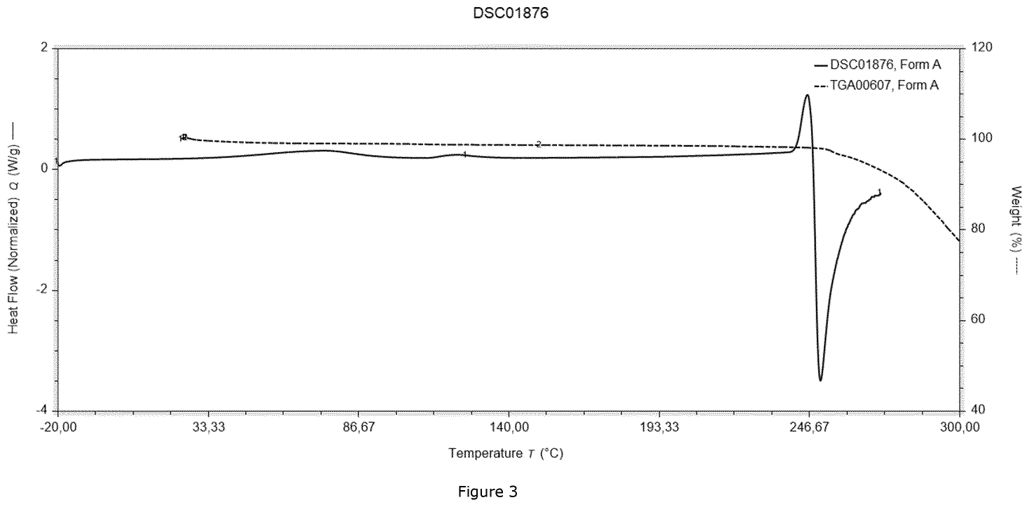

FIG. 4: DSC and TGA curve of methyl sulfate form B.

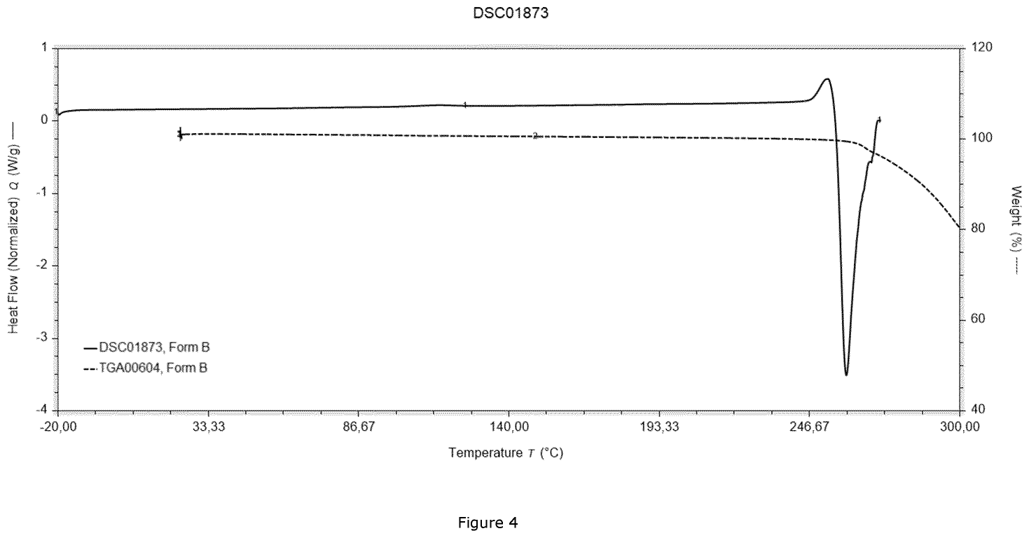

FIG. 5A: The ORTREP drawing of the absolute crystal structure of form A. The asymmetric unit cell consists of one 1-(8-bromopyrido[2,3-e][1,2,4]triazolo[4,3-a]pyrazin-4-yl)-N-methylazetidin-3-amine molecule, one water and one methyl sulphate ion.

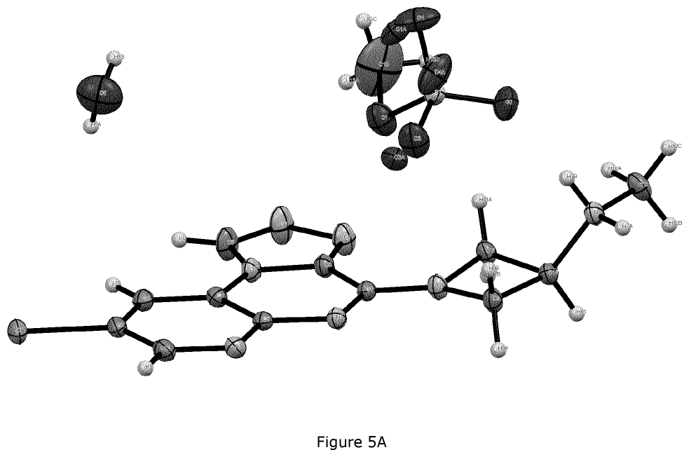

FIG. 5B: The ORTREP drawing of the absolute crystal structure of form B of. The asymmetric unit cell consists of one 1-(8-bromopyrido[2,3-e][1,2,4]triazolo[4,3-a]pyrazin-4-yl)-N-methylazetidin-3-amine molecule and one methyl sulphate ion.

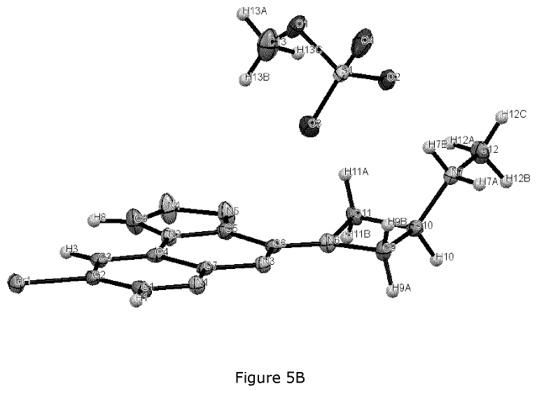

FIG. 6: ¹³C CP/MAS NMR (14.1 T) spectrum of methyl sulfate form A.

FIG. 7: ¹³C CP/MAS NMR (14.1 T) spectrum of methyl sulfate form B.

FIG. 8: XRPD pattern for ethyl sulfate form A.

FIG. 9: ¹³C CP/MAS NMR spectrum for ethyl sulphate form A.

The crystal parameters from the single crystal structure determination of the 1-(8-bromopyrido[2,3-e][1,2,4]triazolo[4,3-a]pyrazin-4-yl)-N-methylazetidin-3-amine methyl sulfate form A and B may be seen in table 3:

TABLE 3

| Crystal form (Sample) | form A | form B |
|---|---|---|
| Empirical formula | C₁₃H₁₈BrN₇O₅S | C₁₃H₁₆BrN₇O₄S |
| Formula weight | 464.31 | 446.30 |
| Temperature/K | 120.00(10) | 120.00(10) |
| Crystal system | monoclinic | triclinic |
| Space group | P2₁/c | P-1 |
| a/Å | 8.66770(10) | 8.3014(4) |
| b/Å | 11.85210(10) | 9.8580(3) |
| c/Å | 17.8714(2) | 11.6328(4) |
| α/° | 90 | 112.203(3) |
| β/° | 93.2860(10) | 91.056(3) |
| γ/° | 90 | 93.547(3) |
| Volume/Å³ | 1832.92(3) | 878.80(6) |
| Z | 4 | 2 |
| ρ_{calc}g/cm³ | 1.683 | 1.687 |
| μ/mm⁻¹ | 4.513 | 2.494 |
| F(000) | 944.0 | 452.0 |
| Crystal size/mm³ | 0.16 × 0.14 × 0.1 | 0.2 × 0.07 × 0.05 |
| Radiation | Cu Kα (λ = 1.54184) | Mo Kα (λ = 0.71073) |
| 2Θ range for data | 8.958 to 147.926 | 6.344 to 59.53 |
| Index ranges | −10 ≤ h ≤ 10, −14 ≤ k ≤ 14, − | −11 ≤ h ≤ 11, −13 ≤ k ≤ 13, − |
| Reflections collected | 33786 | 39620 |
| Independent reflections | 3707 [Rᵢₙₜ = 0.0355, R_{sigma} | 4721 [Rᵢₙₜ = 0.0986, R_{sigma} = |
| Data/restraints/parameters | 3707/0/279 | 4721/0/237 |
| Goodness-of-fit on F² | 1.033 | 1.087 |
| Final R indexes [I >= 2σ (I)] | R₁ = 0.0304, | R₁ = 0.0486, |
| | wR₂ = 0.0745 | wR₂ = 0.0739 |
| Final R indexes [all data] | R₁ = 0.0327, | R₁ = 0.0863, |
| | wR₂ = 0.0763 | wR₂ = 0.0863 |
| Largest diff. peak/hole/e | 0.80/−0.51 | 0.89/−0.57 |

From the data in table 3 it can be concluded that form A is a monoclinic monohydrate and form B is a triclinic anhydrate.

Thus, in one embodiment the invention relates to the 1-(8-bromopyrido[2,3-e][1,2,4]triazolo[4,3-a]pyrazin-4-yl)-N-methylazetidin-3-amine methyl sulfate salt.

In another embodiment the invention relates to crystalline 1-(8-bromopyrido[2,3-e][1,2,4]triazolo[4,3-a]pyrazin-4-yl)-N-methylazetidin-3-amine methyl sulfate monohydrate characterized by one or more XRPD reflections at approximately (° 2θ) 8.9, 12.3 and/or 15.7 (±0.2 degrees).

In a further embodiment the invention relates to a crystalline 1-(8-bromopyrido[2,3-e][1,2,4]triazolo[4,3-a]pyrazin-4-yl)-N-methylazetidin-3-amine methyl sulfate monohydrate, characterized by one or more XRPD reflections at approximately (° 2θ) 8.9, 12.3, 15.7, 16.5, 21.1, 22.4, 23.7, 25.8, 26.0 and/or 29.8 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, characterized by one or more XRPD reflections at approximately 8.9, 12.3 and 15.7 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, characterized by one or more XRPD reflections at approximately (° 2θ) 8.9, 12.3, 15.7, 16.5, 21.1, 22.4, 23.7, 25.8, 26.0 and 29.8 (±0.2 degrees).

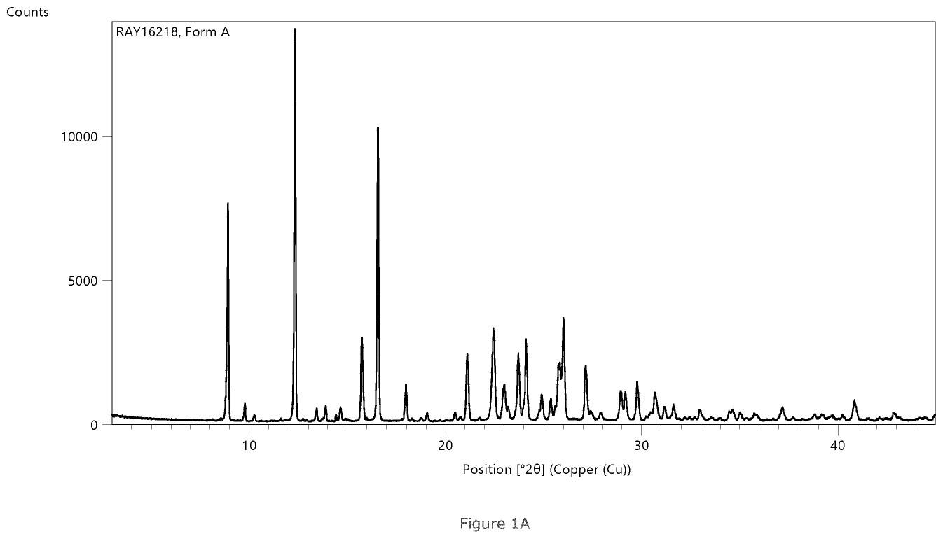

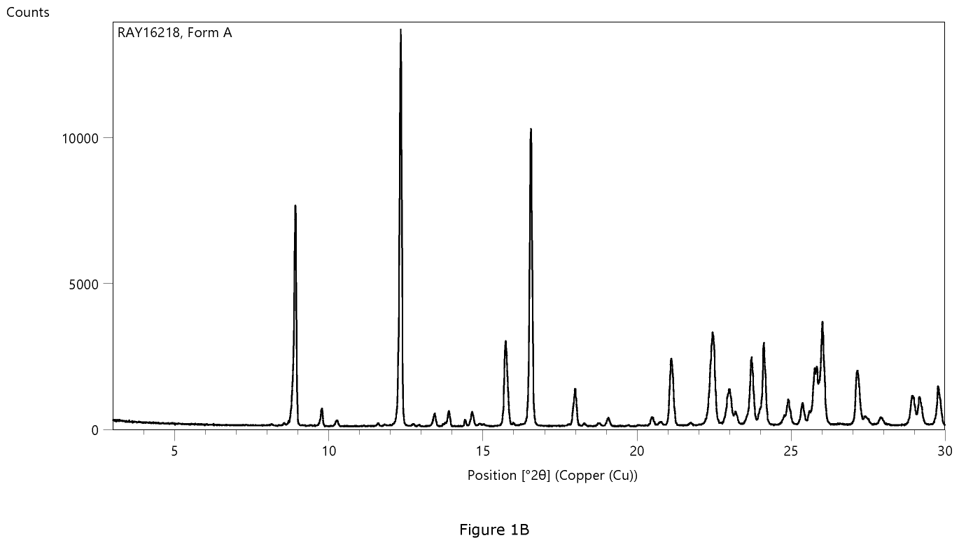

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, wherein the crystalline compound has an XRPD pattern essentially similar to the XRPD pattern in FIG. 1.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, wherein the crystalline compound has an XRPD pattern according to the XRPD pattern in FIG. 1.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, wherein the crystalline compound is characterized by a solid state ¹³C CP/MAS NMR spectrum with peaks at one or more of 149.0, 147.3, 127.8, 119.0, 59.0, 48.9 and/or 33.7 ppm+0.2 ppm.

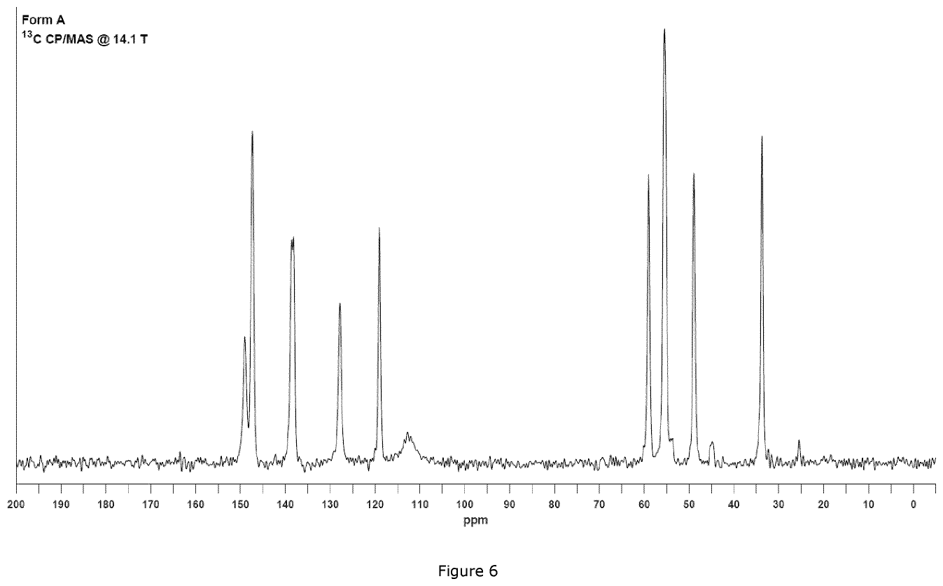

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate characterized by having a ¹³C CP/MAS NMR spectrum essentially similar to the ¹³C CP/MAS NMR spectrum in FIG. 6.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate characterized by having a ¹³C CP/MAS NMR spectrum according to the ¹³C CP/MAS NMR spectrum in FIG. 6.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate characterized by having the above ¹³C CP/MAS NMR spectrum and further characterized by one or more XRPD reflections at approximately (° 2θ) 8.9, 12.3, 15.7, 16.5, 21.1, 22.4, 23.7, 25.8, 26.0 and 29.8 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, wherein the crystalline compound is characterized by having the single-crystal X-Ray crystallography (SXRC) parameters as shown in table 2 for form A.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate which has a DSC curve comprising an endothermic-exothermic event with an onset at 242.2±2° C. and a corresponding weight loss in the TGA curve.

In a further embodiment the invention relates to crystalline 1-(8-bromopyrido[2,3-e][1,2,4]triazolo[4,3-a]pyrazin-4-yl)-N-methylazetidin-3-amine methyl sulfate monohydrate, wherein the molar ratio of 1-(8-bromopyrido[2,3-e][1,2,4]triazolo[4,3-a]pyrazin-4-yl)-N-methylazetidin-3-amine to methyl sulfuric acid is in the range of from 1:0.8 to 1:1.2, and preferably is approximately 1:1

In another embodiment the invention relates to crystalline 1-(8-bromopyrido[2,3-e][1,2,4]triazolo[4,3-a]pyrazin-4-yl)-N-methylazetidin-3-amine methyl sulfate anhydrate characterized by one or more XRPD reflections at approximately (° 2θ) 8.1, 14.9 and/or 26.5 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, characterized by one or more XRPD reflections at approximately (° 2θ) 8.1, 14.3, 14.9, 17.6, 20.1, 21.5, 22.1 and/or 26.5 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, characterized by one or more XRPD reflections at approximately 8.1, 14.9 and 26.5 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, characterized by one or more XRPD reflections at approximately (° 2θ) 8.1, 14.3, 14.9, 17.6, 20.1, 21.5, 22.1 and 26.5 (±0.2 degrees).

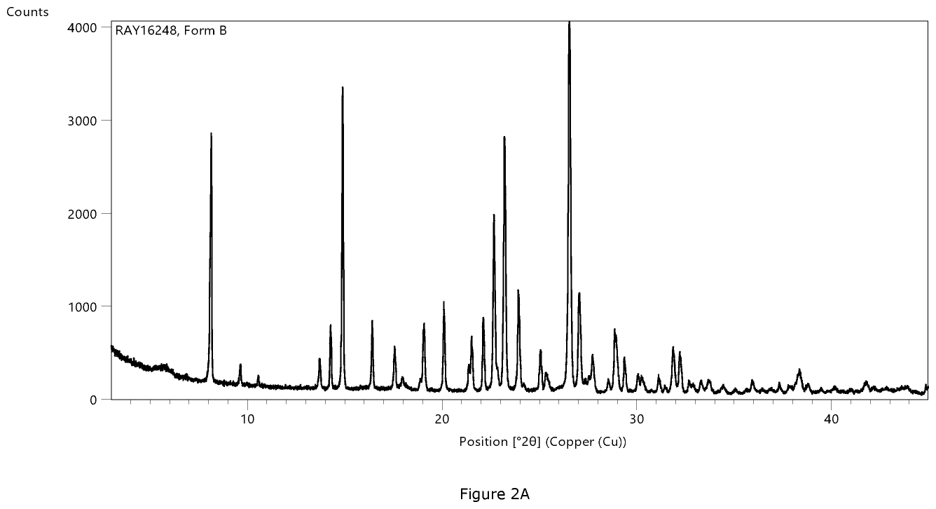

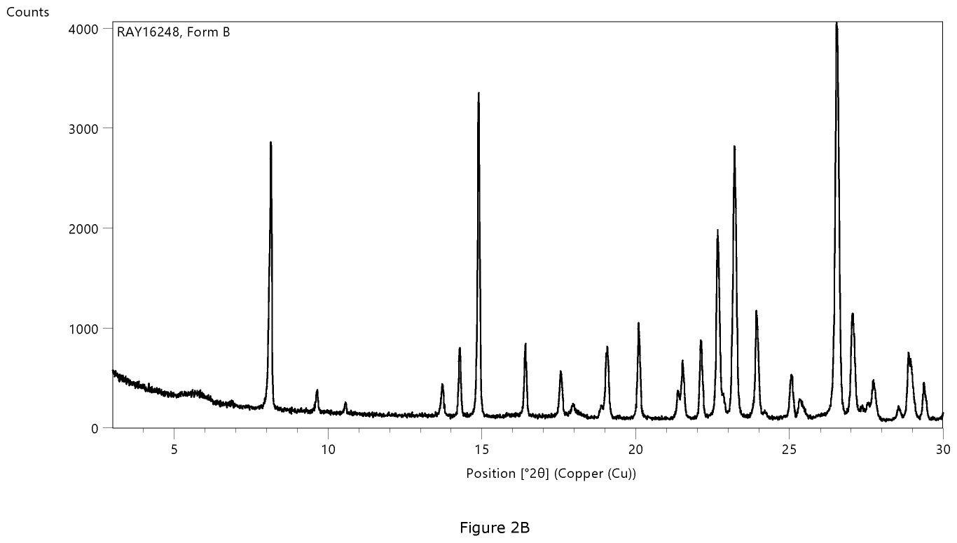

In a further embodiment, the invention relates to crystalline 1-(8-bromopyrido[2,3-e][1,2,4]triazolo[4,3-a]pyrazin-4-yl)-N-methylazetidin-3-amine methyl sulfate anhydrate, wherein the crystalline compound has an XRPD pattern essentially similar to the XRPD pattern in FIG. 2.

In a further embodiment, the invention relates to crystalline 1-(8-bromopyrido[2,3-e][1,2,4]triazolo[4,3-a]pyrazin-4-yl)-N-methylazetidin-3-amine methyl sulfate anhydrate, wherein the crystalline compound has an XRPD pattern according to the XRPD pattern in FIG. 2.

In a further embodiment, the invention relates to crystalline 1-(8-bromopyrido[2,3-e][1,2,4]triazolo[4,3-a]pyrazin-4-yl)-N-methylazetidin-3-amine methyl sulfate anhydrate, wherein the crystalline compound is characterized by a solid state ¹³C CP/MAS NMR spectrum with peaks at one or more of 148.1, 146.4, 128.6, 119.9, 60.3, 48.0 and/or 32.2 ppm+0.2 ppm.

In a further embodiment, the invention relates to crystalline 1-(8-bromopyrido[2,3-e][1,2,4]triazolo[4,3-a]pyrazin-4-yl)-N-methylazetidin-3-amine methyl sulfate anhydrate, wherein the crystalline compound is characterized by a solid state ¹³C CP/MAS NMR spectrum with peaks at one or more of 148.1, 146.4, 128.6, 119.9, 60.3, 48.0, and 32.2 ppm+0.2 ppm.

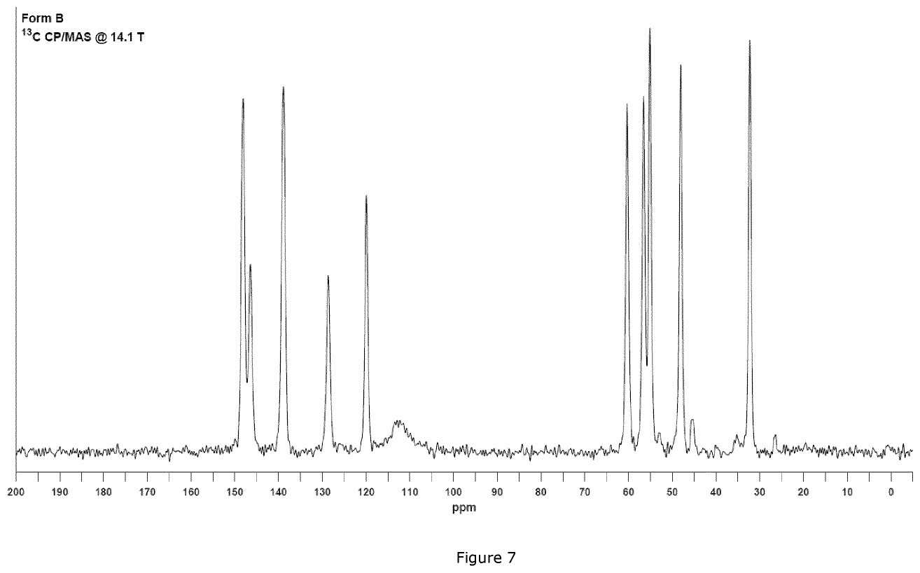

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate characterized by having a ¹³C CP/MAS NMR spectrum essentially similar to the ¹³C CP/MAS NMR spectrum in FIG. 7.

In a further embodiment, the invention relates to crystalline 1-(8-bromopyrido[2,3-e][1,2,4]triazolo[4,3-a]pyrazin-4-yl)-N-methylazetidin-3-amine methyl sulfate anhydrate characterized by having a ¹³C CP/MAS NMR spectrum according to the ¹³C CP/MAS NMR spectrum in FIG. 7.

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, characterized by having the above ¹³C CP/MAS NMR spectrum and further characterized by one or more XRPD reflections at approximately (° 2θ) 8.1, 14.3, 14.9, 17.6, 20.1, 21.5, 22.1 and/or 26.5 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, wherein the crystalline compound is characterized by having the single-crystal X-Ray crystallography (SXRC) parameters as shown in table 1 for form B.

In a further embodiments the invention relates to crystalline 1-(8-bromopyrido[2,3-e][1,2,4]triazolo[4,3-a]pyrazin-4-yl)-N-methylazetidin-3-amine methyl sulfate anhydrate, which has a DSC curve comprising an endothermic event with an onset value at about consisting of a not separated endothermic-exothermic event with an onset at 247.6±2° C. and a corresponding weight loss in the TGA curve.

In a further embodiment the invention relates to crystalline 1-(8-bromopyrido[2,3-e][1,2,4]triazolo[4,3-a]pyrazin-4-yl)-N-methylazetidin-3-amine methyl sulfate anhydrate, characterized in that the molar ratio of 1-(8-bromopyrido[2,3-e][1,2,4]triazolo[4,3-a]pyrazin-4-yl)-N-methylazetidin-3-amine to methyl sulfuric acid is in the range of from 1:0.8 to 1:1.2, and preferably is approximately 1:1.

In a further embodiment the invention relates to crystalline 1-(8-bromopyrido[2,3-e][1,2,4]triazolo[4,3-a]pyrazin-4-yl)-N-methylazetidin-3-amine ethyl sulfate characterized by one or more XRPD reflections at approximately, characterized by one or more XRPD reflections at approximately 8.1, 14.6 and/or 23.4 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine ethyl sulfate, characterized by one or more XRPD reflections at approximately (° 2θ) 8.1, 14.6, 16.3, 17.2, 18.8, 19.0, 20.1, 21.7, 23.4 and/or 26.8 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine ethyl sulfate, characterized by one or more XRPD reflections at approximately 8.1, 14.6 and 23.4 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine ethyl sulfate, characterized by one or more XRPD reflections at approximately (° 2θ) 8.1, 14.6, 16.3, 17.2, 18.8, 19.0, 20.1, 21.7, 23.4 and 26.8 (±0.2 degrees).

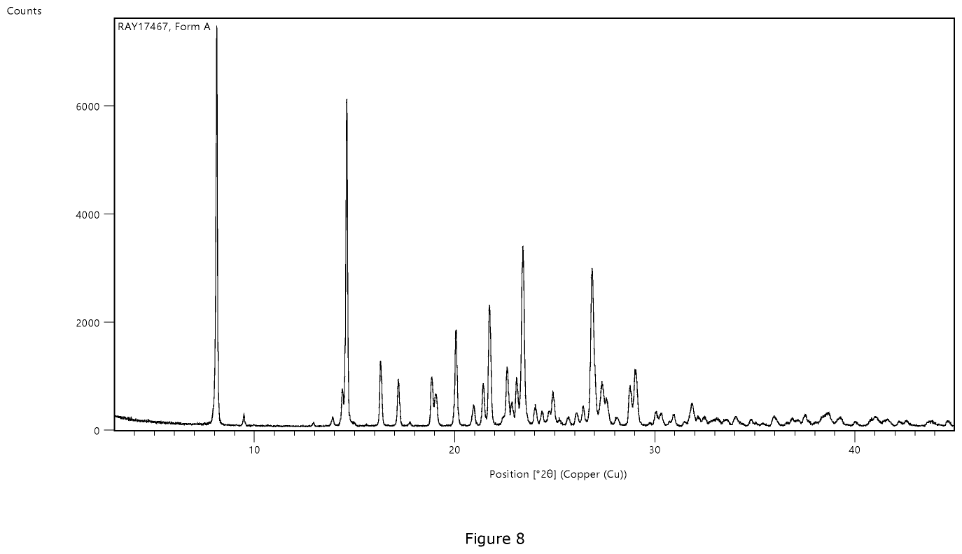

In a further embodiment the invention relates to crystalline 1-(8-bromopyrido[2,3-e][1,2,4]triazolo[4,3-a]pyrazin-4-yl)-N-methylazetidin-3-amine ethyl sulfate, wherein the crystalline compound has an XRPD pattern essentially similar to the XRPD pattern in FIG. 8.

In a further embodiment the invention relates to crystalline 1-(8-bromopyrido[2,3-e][1,2,4]triazolo[4,3-a]pyrazin-4-yl)-N-methylazetidin-3-amine ethyl sulfate, wherein the crystalline compound has an XRPD pattern according to the XRPD pattern in FIG. 8.

In a further embodiment the invention relates to crystalline 1-(8-bromopyrido[2,3-e][1,2,4]triazolo[4,3-a]pyrazin-4-yl)-N-methylazetidin-3-amine ethyl sulfate, wherein the crystalline compound is characterized by a solid state ¹³C CP/MAS NMR spectrum with peaks at one or more of 147.7, 145.8, 139.6, 138.7, 128.9, 119.5, 109.8, 63.2, 59.3, 56.6, 48.2, 31.8 and/or 17.0 ppm+0.2 ppm.

In a further embodiment the invention relates to crystalline 1-(8-bromopyrido[2,3-e][1,2,4]triazolo[4,3-a]pyrazin-4-yl)-N-methylazetidin-3-amine ethyl sulfate, wherein the crystalline compound is characterized by a solid state ¹³C CP/MAS NMR spectrum with peaks at one or more of 147.7, 145.8, 139.6, 138.7, 128.9, 119.5, 109.8, 63.2, 59.3, 56.6, 48.2, 31.8 and 17.0 ppm+0.2 ppm.

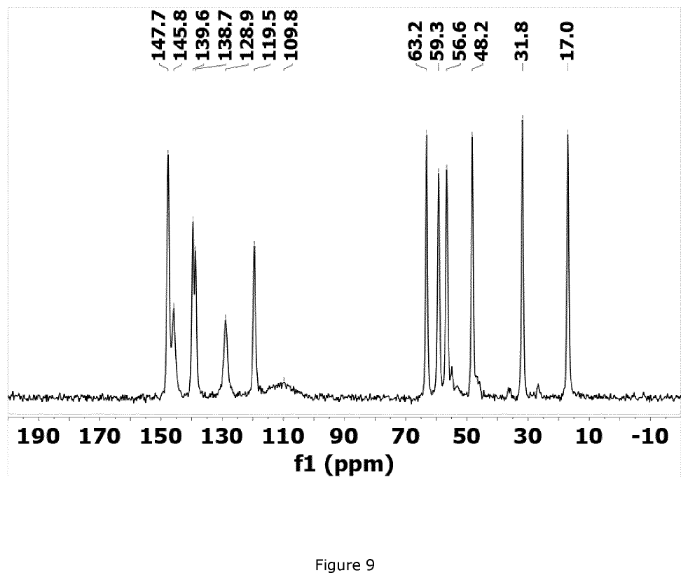

In a further embodiment the invention relates to crystalline 1-(8-bromopyrido[2,3-e][1,2,4]triazolo[4,3-a]pyrazin-4-yl)-N-methylazetidin-3-amine ethyl sulfate, characterized by having a ¹³C CP/MAS NMR spectrum essentially similar to the ¹³C CP/MAS NMR spectrum in FIG. 9.

In a further embodiment the invention relates to crystalline 1-(8-bromopyrido[2,3-e][1,2,4]triazolo[4,3-a]pyrazin-4-yl)-N-methylazetidin-3-amine ethyl sulfate, characterized by having a ¹³C CP/MAS NMR spectrum according to the ¹³C CP/MAS NMR spectrum in FIG. 9.

In additional embodiments the invention relates to pharmaceutical composition comprising a crystalline compound as described above and a pharmaceutically acceptable carrier.

In an additional embodiment the invention relates to a pharmaceutical composition as above for use in the treatment of disease selected from atopic dermatitis, itch, pruritus, and various forms of urticaria.

In a particular embodiment the invention relates to a pharmaceutical composition as above wherein the forms of urticaria include chronic idiopathic urticaria subtypes, such as cholinergic urticaria.
1-(8-bromopyrido[2,3-e][1,2,4]triazolo[4,3-a]pyrazin-4-yl)-N-methylazetidin-3-amine methyl sulfate may be made by crystallization from methanol in presence of sulfuric acid.

The crystalline forms of the invention may be prepared from 1-(8-bromopyrido[2,3-e][1,2,4]triazolo[4,3-a]pyrazin-4-yl)-N-methylazetidin-3-amine in the form of the methyl sulfate salt by recrystallization from a suitable solvent, such as a lower alcohol (such as methanol, ethanol, propanol and mixtures thereof), acetone, acetonitrile, lower alkyl acetate (such as ethyl acetate and propyl acetate), tetrahydrofuran and mixtures of these solvents with various amounts of water, and/or liquid hydrocarbons (such as hexane and heptane) at rt or cooled to temperatures below rt such as temperatures below 0° C., suitably—18° C.

For the formation of form B, 1-(8-bromopyrido[2,3-e][1,2,4]triazolo[4,3-a]pyrazin-4-yl)-N-methylazetidin-3-amine methyl sulfate hydrate is suspended in methanol followed by heating of the reaction and once most of the 1-(8-bromopyrido[2,3-e][1,2,4]triazolo[4,3-a]pyrazin-4-yl)-N-methylazetidin-3-amine methyl sulfate is dissolved, the solution was allowed to cool to room temperature.

For the formation of form A, 1-(8-bromopyrido[2,3-e][1,2,4]triazolo[4,3-a]pyrazin-4-yl)-N-methylazetidin-3-amine methyl sulfate is suspended in the solvent (such as 2-propanol) followed by heating of the reaction and once most of the 1-(8-bromopyrido[2,3-e][1,2,4]triazolo[4,3-a]pyrazin-4-yl)-N-methylazetidin-3-amine methyl sulfate is dissolved, a small amount of water is added, and the solution was allowed to cool to room temperature. The amount of water to the amount of solvent is suitably in the range 1:2 to 1:6, suitably 1:4.

This can be conveniently obtained by using methyl hydrogen sulfate in methanol to deprotect the Boc group. Solvents for this crystallization include methanol and water, and isopropyl alcohol as antisolvent. It can also be obtained conveniently directly from the reaction mixture (methanol as solvent) by cooling the reaction mixture to room temperature followed by filtration.

The free base of the compound of formula (I) including crystalline forms can be conveniently obtained from the methyl sulfate salt by stirring the methyl sulfate salt in acetone in the presence of triethylamine.

The hemisuccinate of the compound of formula (I) including crystalline forms can be obtained from the free base by stirring a mixture of the free base and succinic acid in an appropriate solvent such as 1-propanol optionally with water.

The hemisuccinate of the compound of formula (I) including crystalline forms salt can be prepared from the methyl sulfate salt by dissolving the methyl sulfate salt in water, optionally in the presence of another solvent such as acetone, followed by the addition of disodium succinate (0.55 eq). The hemisuccinate is then precipitated by either cooling or the addition of an antisolvent, or both.

The ethyl sulfate salt of the compound of formula (I) including crystalline forms can be conveniently prepared from the free base by stirring with a solution of ethyl hydrogen sulfate in ethanol, with the addition of water and isopropanol.

The ethyl sulfate salt of the compound of formula (I) including crystalline forms can be conveniently converted to the hemisuccinate of the compound of formula (I) by dissolving the ethyl sulfate salt in water, followed by the addition of disodium succinate, followed by optional addition of an antisolvent (for example acetone) and with optional cooling.

The methane sulfonate salt of the compound of formula (I) including crystalline forms can be conveniently converted to the free base of the compound of formula (I) by treating the methanol sulfate salt with acetonitrile and triethylamine. The product is isolated by filtration, and then washed with suitable solvents, for example isopropanol and water.

The methane sulfonate salt of the compound of formula (I) including crystalline forms can be conveniently prepared in a number of different ways. One is the treatment of the free base with methane sulfonic acid in a suitable solvent, such as acetonitrile.

The methane sulfonate salt of the compound of formula (I) can also be obtained from precursor (II) (the Boc-protected analogue of the free base).

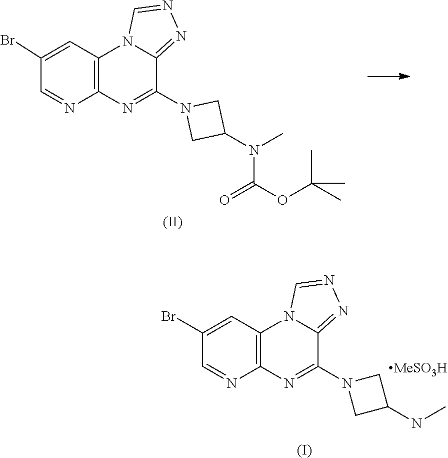 [SMILES: CN(C(=O)OC(C)(C)C)C1CN(c2nc3ncc(Br)cc3n3cnnc23)C1 ; "·MeSO3H" ; "(II)" ; CNC1CN(c2nc3ncc(Br)cc3n3cnnc23)C1 ; "(I)"]

In this process, the compound of formula (II) is stirred with methane sulfonic acid in methanol. The solution was concentrated, followed by the addition of water and isopropanol. The suspension was cooled, and the product was obtained by filtration.

The methane sulfonate salt can also be obtained in a one-pot process from an earlier intermediate, the compound of formula (III).

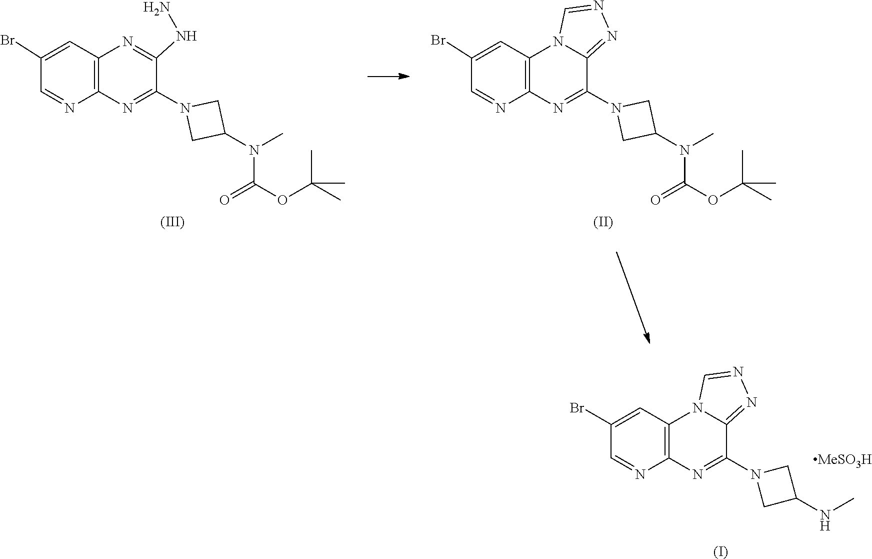

In this process, the intermediate (III) is first treated with trimethyl orthoformate in methanol giving the intermediate (II), which is then treated with methane sulfonic acid in a similar manner to the previous example.

The methane sulfonate salt of the compound of formula (I) including crystalline forms thereof can also be conveniently converted to the hemisuccinate salt by slurrying the methane sulfonate salt in water, optionally water and acetone. A solution of disodium succinate in water was added, followed by seeding. After the transformation was complete, the mixture was cooled, and filtered to provide the hemisuccinate salt.

A further aspect of the present invention is directed to a pharmaceutical composition comprising the crystalline compound of the present invention and at least one pharmaceutically acceptable excipient. The pharmaceutical composition may be an oral dosage form, preferably a tablet and/or capsule.

In addition, the present invention relates to the use of the crystalline compound of the present invention for the preparation of a solid medicament.

In another embodiment the present invention relates to solid pharmaceutical compositions comprising an effective amount of the crystalline compound of the present invention and a pharmaceutically acceptable carrier as well as to processes of preparing the same. Moreover, the present invention is directed to the pharmaceutical composition of the present invention and/or the crystalline compound of the present invention for use in the treatment of any of the disease or disorders mentioned in U.S. Pat. No. 9,586,959, including diseases and disorders such as atopic dermatitis (AD), itch, pruritus and any of the various types of urticaria.

The pharmaceutical compositions of the present invention comprising the crystalline compound of the present invention may further comprise one or more pharmaceutically acceptable excipients. Such excipients are preferably selected from the group consisting of diluents, sweeteners, buffering agents, glidants, flowing agents, flavouring agents, lubricants, preservatives, surfactants, wetting agents, binders, disintegrants and thickeners. Other excipients known in the field of pharmaceutical compositions may also be used. Furthermore, the pharmaceutical composition may comprise a combination of two or more excipients also within one of the members of the above-mentioned group.

Suitable binders which can be used for the pharmaceutical compositions of the present invention comprising the crystalline compound of the present invention, further comprise e.g. alkylcelluloses such as methylcellulose, hydroxyalkylcelluloses such as hydroxymethylcellulose, hydroxyethylcellulose, hydroxypropylcellulose and hydroxybutylcellulose, hydroxyalkylalkylcelluloses such as hydroxyethylmethylcellulose and hydroxypropylmethylcellulose, carboxyalkylcelluoses such as carboxymethylcellulose, alkali metal salts of carboxyalkylcelluloses such as sodium carboxymethylcellulose, carboxyalkylalkylcelluloses such as carboxymethylethylcellulose, carboxyalkylcellulose esters, starches, modified starches such as sodium carboxymethyl starch, pectins, chitin derivatives such as chitosan, heparin and heparinoids, polysaccharides such as alginic acid, alkali metal and ammonium salts thereof, carrageenans, galactomannans, tragacanth, agar-agar, gum arabic, guar gum and xanthan gum, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, polyalkylene oxides such as polyethylene oxide and polypropylene oxide and copolymers of ethylene oxide and propylene oxide, e.g. poloxamers and poloxamines, copovidone.

Suitable diluents which can be used for the pharmaceutical compositions of the present invention comprising the crystalline compound of the present invention further comprise e.g. calcium carbonate, dibasic calcium phosphate, dibasic calcium phosphate dihydrate, tribasic calcium phosphate, calcium sulphate, microcrystalline cellulose including silicified microcrystalline cellulose, powdered cellulose, dextrates, dextrin, dextrose excipient, fructose, kaolin, lactitol, lactose anhydrous, lactose monohydrate, mannitol, sorbitol, starch, modified starch, sodium chloride, sucrose, compressible sugar, confectioner's sugar, a spray-dried mixture of lactose monohydrate and microcrystalline cellulose (75:25), commercially available as Microcelac®, a co-processed spray-dried mixture of microcrystalline cellulose and colloidal silicon dioxide (98:2), commercially available as Prosolv®.

Suitable glidants which can be used for the pharmaceutical compositions of the present invention comprising the crystalline compound of the present invention further comprise e.g. talc, colloidal silicon dioxide, starch and magnesium stearate.

Suitable disintegrants which can be used for the pharmaceutical compositions of the present invention comprising the crystalline compound of the present invention further comprise e.g. starch, ion exchange resins, e.g. Amberlite, cross-linked polyvinylpyrrolidone, modified cellulose gum, e.g croscarmellose sodium, sodium starch glycolate, sodium carboxymethylcellulose, sodium dodecyl sulphate, modified corn starch, microcrystalline cellulose, magnesium aluminium silicate, alginic acid, alginate and powdered cellulose.

Suitable lubricants which can also be used for the pharmaceutical compositions of the present invention comprising the crystalline compound of the present invention further comprise e.g. magnesium stearate, calcium stearate, stearic acid, talc, polyethylene glycol, sodium lauryl sulphate and magnesium lauryl sulphate.

Some formulations, e.g. tablets may contain ingredients that have XRPD reflection peaks in the same position or area as the crystalline compound of the invention or have broad peaks. These may hide some of the XRPD pattern or peaks of the crystalline compound of the invention when the XRPD experiment is performed on a formulation comprising the crystalline compound of the invention as opposed to the pure crystalline salt alone. This means that one cannot always see all XRPD reflection peaks of the crystalline compound of the invention when an XRPD experiment is performed on a formulation of the crystalline compound

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which exhibit XRPD reflection peaks including one or more XRPD reflection peaks that overlap with and hide one or more XRPD reflection peaks of the crystalline compound of the invention.

The same issues may arise with solid state ¹³C CP/MAS NMR where for example intense signals from a cellulose component should be expected in the spectral region 60-110 ppm and the peaks from stearate will be seen in the spectral region 15-40 ppm-along with a carbonyl peak around 172 ppm.

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which are characterized by a ¹³C CP/MAS NMR spectrum that may include one or more peaks that overlap with and hide one or more ¹³C CP/MAS NMR peaks of the crystalline compound of the invention.

The absence of other crystalline forms of 1-(8-bromopyrido[2,3-e][1,2,4]triazolo[4,3-a]pyrazin-4-yl)-N-methylazetidin-3-amine methyl sulfate, ethyl sulfate and methane sulfonate can be tested by comparing an XRPD pattern taken of any crystalline form of 1-(8-bromopyrido[2,3-e][1,2,4]triazolo[4,3-a]pyrazin-4-yl)-N-methylazetidin-3-amine or a salt thereof with an XRPD pattern of the crystalline forms of the invention disclosed in the figures herein.

### Description of the Test Methods Used to Characterize the Polymorphic Forms Disclosed Herein

### X-Ray Powder Diffraction (XRPD):

XRPD patterns were collected with a PANalytical X′pert PRO MPD diffractometer using an incident Cu Kα radiation and operating at 45 kV and 40 mA. The XRPD patterns were collected in the 2 theta range from 3 to 45 degrees with a stepsize of 0.0066°, counting time of 148.93 s and in transmission geometry. In the incident beam path, an elliptically graded multilayer mirror together with a 4 mm fixed mask, fixed anti-scatter slit 1° and fixed divergence slits of ½° were placed to line focus the Cu Kα X-rays through the sample and onto the detector. At the diffracted beam path, a long antiscatter extension and antiscatter slits of 2 mm were placed to minimize the background generated by air. Furthermore, Soller slits of 0.02 rad were placed on both the incident and diffracted beam paths to minimize broadening from axial divergence.

The sample was placed on a 3 μm thick foil on a 96 high throughput well plate stage and oscillated in the X direction for better particle statistics. The diffraction patterns were collected using a PIXel RTMS detector with an active length of 3.347° and located 240 mm from the sample.

### Thermo Gravimetric Analysis (TGA):

TGA experiments were conducted using a TGA550 instrument from TA Instruments. About 1-10 mg of sample was loaded into a ceramic pan for the measurements. The sample temperature was ramped from 25 to 500° C. at 10° C./min. Nitrogen was used as the purge gas at a flow rate of 50 mL/min.

### Differential Scanning Calorimetry (DSC):

DSC: Heating rate of 10° C./min under a nitrogen atmosphere. About 1-2 mg of sample was loaded into an open aluminum pan for the measurements. Instrument Q20 from TA Instruments.

### Single-Crystal X-Ray Diffraction:

Data were collected using a SuperNova, Dual diffractometer with an Atlas CCD area detector (Temperature: 120 (2) K; Cu Kα Radiation λ=1.5418 Å; data collection method: Ω scans). Further details can be found in Error! Reference source not found. Program(s) used to solve structure: CrysAlisPro, Agilent Technologies, Version 1.171.37.34 (release 22-05-2014 CrysAlis171.NET), ShelXL (Sheldrick, 2008) used to refine structure and Olex2 (Dolomanov et al., 2009) for ORTEP drawings.

### Solid-State NMR Spectroscopy:

¹³C CP/MAS NMR spectra were recorded at 298 K on a Bruker Avance III HD 600 NMR spectrometer (14.1 T) equipped with a 4 mm double-tuned (1H-¹³C) CMP probe using a contact time of 6 ms, a recycle delay of 64 s, spin-rate of 14.1 KHz, 256 scans and high-power 1H decoupling during the acquisition time of 45.9 ms. Prior to Fourier Transformation the time domain data (Free Induction Decay) was apodized by Lorentzian linebroadening of 5 Hz. All spectra are referenced to the chemical shift of the carbonyl group in α-glycine at 176.5 ppm (external sample).

The given error ranges in this application for the spectroscopic characteristics, including those in the claims, may be more or less depending on factors well known to a person skilled in the art of spectroscopy and may for example depend on sample preparation, such as particle size distribution, or if the crystal form is part of a formulation, on the composition of the formulation, as well as instrumental fluctuations, and other factors.

### EXAMPLES

### Example 1

### Form A

11 mg of 1-(8-bromopyrido[2,3-e][1,2,4]triazolo[4,3-a]pyrazin-4-yl)-N-methylazetidin-3-ammonium methyl sulfate was added to a test tube or 4 ml glass vial and 4 mL of 2-propanol followed by 0.2 mL water were added. The solution was heated with a heat gun until all solid material was visually dissolved. The solution was then filtered with a 0.45 μm syringe filter. The filtered solution was placed on the table in the fume cup with a loose lid for crystallization during slowly evaporation of the solvent system. The resulting crystals were isolated by filtration. The XRPD of the crystalline material is shown in FIG. 1.

### Example 2

### Form B

11 mg of 1-(8-bromopyrido[2,3-e][1,2,4]triazolo[4,3-a]pyrazin-4-yl)-N-methylazetidin-3-ammonium methyl sulfate was added to a test tube or 4 ml glass vial and 4 mL of EtOH followed by 0.2 mL water were added. The solution was heated with a heat gun until all solid material was visually dissolved. The solution was then filtered with a 0.45 μm syringe filter. The filtered solution was placed on the table in the fume cup with a loose lid for crystallization during slowly evaporation of the solvent system. The resulting crystals were isolated by filtration. The XRPD of the crystalline material is shown in FIG. 2.

### Example 3

Preparation of 1-(8-bromopyrido[2,3-e][1,2,4]triazolo[4,3-a]pyrazin-4-yl)-N-methylazetidin-3-ammonium methyl sulfate (Form B)

To methanol (33 ml) was added concentrated sulfuric acid (6.5 g) portion-wise over 10 min. The initial temperature was 15° C., and despite the exothermic reaction, the temperature never exceeded 37° C. Following the addition, the mixture was heated to a mild reflux (internal temperature 64° C., and after 1 h at reflux the solution was allowed to cool to 20° C. and was stirred at that temperature until use.

To Compound (III) (15 g) was added methanol (150 ml) and the mixture was stirred. The mixture was heated to 50° C. over 15 min. An aliquot of the methanol solution of methyl hydrogen sulfate previously prepared (0.24 ml) was added, and after 10 minutes, trimethyl orthoformate (8.9 ml) was added dropwise over 30 min. After approximately 2.3 ml was added the mixture turned orange. After addition was complete the reaction mixture was stirred at 50° C. for approximately 18 h. The orange suspension became a very thick red suspension, which then slowly turned to a yellow suspension overnight. At the end of the 18 h the remainder of the solution of methyl hydrogen sulfate in methanol was added dropwise over 30 min. By the end of the addition most of the solid had dissolved. The mixture was stirred for 8 h at 50° C., after which the temperature was reduced to 2° C. over 2 h, and the suspension was stirred overnight. The suspension was filtered, and the residue was washed with methanol (45 ml). The residue was dried on the filter, then dried under vacuum at 35° C. over the weekend to afford the desired methyl sulfate salt as a yellow solid (13.4 g, approximately 92% yield, >99% purity).

### Example 4

Preparation of 1-(8-bromopyrido[2,3-e][1,2,4]triazolo[4,3-a]pyrazin-4-yl)-N-methylazetidin-3-ammonium ethyl sulfate

A solution of sulfuric acid (12 ml) and ethanol (250 ml) was heated at 50° C. overnight. The solution was then concentrated to approximately 75 ml, and then allowed to cool to room temperature.

In a separate flask the free base of 1-(8-bromopyrido[2,3-e][1,2,4]triazolo[4,3-a]pyrazin-4-yl)-N-methylazetidin-3-amine (25 g) was added, followed by the ethanol solution prepared earlier. Water (375 ml) was added, followed by isopropanol (500 ml), and the mixture was stirred. When the crystallization was complete, the product was isolated by filtration, and the filter cake was washed with isopropanol. Yield approximately 82%, purity approximately 95%.

### Example 5

### Preparation of 1-(8-bromopyrido[2,3-e][1,2,4]triazolo[4,3-a]pyrazin-4-yl)-N-methylazetidin-3-ammonium methane sulfonate

A mixture of tert-butyl N-[1-(12-bromo-2,4,5,8,10-pentazatricyclo[7.4.0.02,6]trideca-(9),3,5,7,10,12-hexaen-7-yl) azetidin-3-yl]-N-methyl-carbamate (50 g) in methanol (750 ml) was stirred, and methane sulfonic acid (22.4 ml) was added dropwise over approximately 7 minutes. The mixture was stirred at 50° C. overnight. The mixture was allowed to cool, and the mixture was concentrated under reduced pressure (approximately 500 ml solvent was removed). The mixture was filtered, and the residue was transferred to a suitable flask. Water (500 ml) was added, followed by activated charcoal (10 g). The mixture was seated at 50° C. for approximately 30 min. The carbon was removed by filtration. The filtrate was transferred to a suitable flask, and isopropanol (1500 ml) was added. The slurry was stirred overnight at room temperature. The mixture was filtered, and the filter cake was washed with isopropanol. The filter cake was dried under suction on the filter, and then dried under vacuum overnight. Yield approximately 76%, purity approximately 99%.

### Example 6

Preparation of the Free Base from the Methyl Sulfate Salt

The 1-(8-bromopyrido[2,3-e][1,2,4]triazolo[4,3-a]pyrazin-4-yl)-N-methylazetidin-3-amine methyl sulfate salt (1200 g) was suspended with stirring in acetone (17.5 L). Triethylamine (750 ml) was added, and the mixture was stirred overnight at room temperature. The solid product was isolated by filtration, and was washed with acetone, and was dried under vacuum. Yield approximately 95%, purity>90%.

### Example 7

Preparation of the Hemisuccinate Form F from the Methyl Sulfate Salt

The 1-(8-bromopyrido[2,3-e][1,2,4]triazolo[4,3-a]pyrazin-4-yl)-N-methylazetidin-3-amine methyl sulfate salt (20 g) was added to a suitable flask, followed by water (180 ml) and acetone (40 ml). The reaction mixture was filtered, and the filtrate was transferred to a suitable reactor. A solution of disodium succinate (4 g) in water (24 ml) was added slowly over 60 min. The solution was then seeded, and acetone (160 ml) was added over approximately 60 min. Precipitation was observed after approximately 20 ml acetone had been added. When the addition of acetone was complete, the mixture/slurry was stirred overnight at 40° C. The reaction mixture was then cooled to 5° C. over 2 h, prior to being filtered. The filter cake was washed with acetone before drying under vacuum at 50° C. overnight. Yield approximately 85%, purity approximately 99%.

### Example 8

Preparation of the Hemisuccinate Form F from the Ethyl Sulfate Salt

To the 1-(8-bromopyrido[2,3-e][1,2,4]triazolo[4,3-a]pyrazin-4-yl)-N-methylazetidin-3-amine ethyl sulfate salt (5 g) was added water (50 ml) with stirring, and the suspension was heated to 40° C. Full dissolution was not observed, so further water (50 ml) was added, and the suspension was heated to 50° C., and the solid was seen to dissolve. Disodium succinate (480 mg) was added as a solid, and the mixture was seeded. Acetone (100 ml) was added, and the reaction mixture was stirred at 40° C. overnight. The next day the reaction mixture was cooled to 5° C. over 2 h. The slurry was then filtered. The filter cake was washed with acetone, and then dried under vacuum at 50° C. overnight. Yield: approximately 72%, purity>90%.

### Example 9

### Conversion of the Methane Sulfonate Salt to the Free Base

A mixture of the 1-(8-bromopyrido[2,3-e][1,2,4]triazolo[4,3-a]pyrazin-4-yl)-N-methylazetidin-3-amine methane sulfonate salt (4.5 g) and acetonitrile (90 ml) was stirred and cooled to approximately 7° C. Triethylamine (2.2 ml) was added, and the mixture was stirred for approximately 90 min. The mixture was filtered, and the filter cake was washed with acetonitrile. The filter cake was transferred to a suitable container and was slurried in a mixture of isopropanol (38.3 ml) and water (6.75 ml). The slurry was then filtered, and the filter cake was dried overnight under vacuum. Yield: approximately 92%, purity approximately 94%.

### Example 10

### Conversion of Intermediate (II) to the Methane Sulfonate Salt

To the intermediate (II) (50 g) was added methanol (750 ml) and the slurry was stirred using a mechanical stirrer. Methane sulfonic acid (22.4 ml) was slowly added over approximately 7 min, and the reactor temperature was then increased to 50° C. and was stirred at that temperature overnight. The solution was then allowed to cool, and the solvent was reduced under vacuum (approximately 500 ml was removed). Water (500 ml) was added, followed by activated charcoal (10 g) and the mixture was heated to 50° C. After approximately 30 min the suspension was filtered, and the filtrate was returned to a suitable reaction vessel. Isopropanol (1500 ml) was added dropwise, and the resultant slurry was stirred overnight at room temperature. The slurry was cooled using an ice-water bath, and after approximately 30 min the slurry was filtered. The filter cake was washed with isopropanol, and then dried first by suction on the filter, and then under vacuum over approximately 3 days. Yield: approximately 76%, purity>90%.

1.-56. (canceled)

57. Crystalline 1-(8-bromopyrido[2,3-e][1,2,4]triazolo[4,3-a]pyrazin-4-yl)-N-methylazetidin-3-amine methyl sulfate monohydrate characterized by one or more XRPD reflections at approximately (° 2θ) 8.9, 12.3 and/or 15.7 (±0.2 degrees).

58. The crystalline compound according to claim 57, wherein one or more XRPD reflections at approximately (° 2θ) 8.9, 12.3, 15.7, 16.5, 21.1, 22.4, 23.7, 25.8, 26.0 and/or 29.8 (±0.2 degrees).

59. The crystalline compound according to claim 57, wherein one or more XRPD reflections at approximately 8.9, 12.3 and 15.7 (±0.2 degrees).

60. The crystalline compound according to claim 57, wherein one or more XRPD reflections at approximately (° 2θ) 8.9, 12.3, 15.7, 16.5, 21.1, 22.4, 23.7, 25.8, 26.0 and 29.8 (±0.2 degrees).

61. The crystalline compound according to claim 57, wherein the crystalline compound has an XRPD pattern essentially similar to the XRPD pattern in FIG. 1.

62. The crystalline compound according to claim 57, wherein the crystalline compound has an XRPD pattern according to the XRPD pattern in FIG. 1.

63. The crystalline compound according to claim 57, wherein the crystalline compound is characterized by a solid state ¹³C CP/MAS NMR spectrum with peaks at one or more of 149.0, 147.3, 127.8, 119.0, 59.0, 48.9 and/or 33.7 ppm±0.2 ppm.

64. The crystalline compound according to claim 57, wherein the crystalline compound is characterized by a solid state ¹³C CP/MAS NMR spectrum with peaks at one or more of 149.0, 147.3, 127.8, 119.0, 59.0, 48.9 and 33.7 ppm±0.2 ppm.

65. The crystalline compound according to claim 64, wherein having a ¹³C CP/MAS NMR spectrum essentially similar to the ¹³C CP/MAS NMR spectrum in FIG. 6.

66. The crystalline compound according to claim 64, which has a ¹³C CP/MAS NMR spectrum according to the ¹³C CP/MAS NMR spectrum in FIG. 6.

67. The crystalline compound according to claim 64, further characterized by one or more XRPD reflections at approximately (° 2θ) 8.9, 12.3, 15.7, 16.5, 21.1, 22.4, 23.7, 25.8, 26.0 and 29.8 (±0.2 degrees).

68. The crystalline compound according to claim 57, wherein the crystalline compound is characterized by having the single-crystal X-Ray crystallography (SXRC) parameters as shown in table 1 for form A.

69. The crystalline compound according to claim 57, which has a DSC curve comprising an endothermic-exothermic event with an onset at 242.2±2° C. and a corresponding weight loss in the TGA curve.

70. The crystalline compound according to claim 57, wherein the molar ratio of 1-(8-bromopyrido[2,3-e][1,2,4]triazolo[4,3-a]pyrazin-4-yl)-N-methylazetidin-3-amine to methyl sulfuric acid is in the range of from 1:0.8 to 1:1.2, and preferably is approximately 1:1.

71. The crystalline compound according to claim 57, wherein the molar ratio of 1-(8-bromopyrido[2,3-e][1,2,4]triazolo[4,3-a]pyrazin-4-yl)-N-methylazetidin-3-amine methyl sulfate to water is in the range from 1:0.8 to 1:1.2, and preferably is approximately 1:1.

72. Crystalline 1-(8-bromopyrido[2,3-e][1,2,4]triazolo[4,3-a]pyrazin-4-yl)-N-methylazetidin-3-amine methyl sulfate anhydrate characterized by one or more XRPD reflections at approximately (° 2θ) 8.1, 14.9 and/or 26.5 (±0.2 degrees).

73. Crystalline 1-(8-bromopyrido[2,3-e][1,2,4]triazolo[4,3-a]pyrazin-4-yl)-N-methylazetidin-3-amine ethyl sulfate characterized by one or more XRPD reflections at approximately, characterized by one or more XRPD reflections at approximately 8.1, 14.6 and/or 23.4 (±0.2 degrees).

74. A pharmaceutical composition comprising the crystalline compound according to claim 57 or pharmaceutically acceptable salt thereof and a pharmaceutically acceptable carrier.

75. A method for treating atopic dermatitis, comprising:
administering to a subject in need thereof the pharmaceutical composition according to claim 74.
